(19) United States
(12) Patent Application Publication
(10) Pub. No.: US 2013/0237442 A1
(43) Pub. Date: Sep. 12, 2013

(54) Methods and Compositions for Diagnosis of Non-Viable Early Pregnancy

(71) Applicants: THE TRUSTEES OF THE UNIVERSITY OF PENNSYLVANIA, Philadelphia, PA (US); UNIVERSITY OF MIAMI, Miami, FL (US)

(72) Inventors: Kurt Barnhart, Bryn Mawr, PA (US); Ram Datar, Miami, FL (US); Peter Takacs, Miami Beach, FL (US)

(73) Assignees: UNIVERSITY OF MIAMI, Miami, FL (US); THE TRUSTEES OF THE UNIVERSITY OF PENNSYLVANIA, Philadelphia, PA (US)

(21) Appl. No.: 13/785,844

(22) Filed: Mar. 5, 2013

Related U.S. Application Data
(60) Provisional application No. 61/607,813, filed on Mar. 7, 2012.

(51) Int. Cl. C12N 15/10; G01N 33/76; G01N 33/68; C12Q 1/68

(52) U.S. Cl. CPC C12N 15/1072; C12Q 1/6876; G01N 33/76; G01N 33/689
USPC 506/9; 435/6.11; 435/7.92; 506/16; 506/18

(57) ABSTRACT

Methods and compositions are provided for diagnosing an abnormal early pregnancy in a mammalian subject by contacting a biological sample of the subject with a reagent that enables measurement of certain biomarker targets, e.g., human placental lactogen (hPL) and/or human chorionic gonadotropin (hCG). In one embodiment, the mRNA of these biomarkers is measured in a biological sample, e.g., serum. The absolute levels of mRNA or protein levels, a ratio of mRNA to protein levels, or a pattern of multiple biomarker mRNA and/or protein levels or ratios are measured and a relation to the ratio or pattern of expression levels of the same biomarkers in the same biological fluid of a reference or control female mammalian subject having a normal intrauterine pregnancy (IUP) is determined. The presence of, absence of, or changes in expression levels, ratios or patterns of the biomarker(s) in relation to those of the reference or control correlates with a diagnosis of abnormal pregnancy, i.e., miscarriage or ectopic pregnancy. Various reagents for use in kits and panels for such diagnosis include PCR primer-probe sets or ligands, labeled or immobilized, which are capable of detecting the changes in expression or translation of these biomarker targets.

### CROSS-REFERENCE TO RELATED APPLICATIONS

This application claims the benefit of the priority of U.S. Provisional Patent Application No. 61/607,813, filed Mar. 7, 2012. The priority application is incorporated herein by reference.

### STATEMENT REGARDING FEDERALLY SPONSORED RESEARCH OR DEVELOPMENT

This invention was made with government support under Grant No. 5-R01-HD036455 awarded by the National Institutes of Health. The government has certain rights in this invention.

### BACKGROUND OF THE INVENTION

Ectopic Pregnancy (EP) is a clinical condition that occurs when the embryo implants at a site other than in the uterus, typically the fallopian tube. As the fetus grows, this condition becomes life-threatening due to potential tubal rupture and internal hemorrhage. EP affects an estimated 1%-2% of all pregnancies and causes approximately 6% of all pregnancy-related deaths. The incidence of EP is increasing due to a number of factors, and it is now the second-most-common cause of maternal death in the first trimester of pregnancy. Nearly a third of all cases do not exhibit any clinical signs and 9% have no symptoms prior to tubal rupture.

The diagnosis of EP, particularly at an early stage, continues to be a clinical challenge for physicians if the location of the pregnancy is not identified via ultrasound on initial presentation. EP is currently diagnosed using an algorithm based on a combination of serial trans-vaginal ultrasounds and serial detections of the protein biomarker, β-human chorionic gonadotropin (β-hCG, gene name: CGB) levels, in serum. Until accurate diagnosis and treatment, the ectopic pregnancy is at risk of rupture, with a possibility of maternal death. Approximately 50% of patients with this condition initially are misdiagnosed—resulting in significant morbidity and mortality.

There is no good experimental model system for EP and efforts to diagnose EP at an early point in the pregnancy using blood tests have been hampered because of the lack of useful and reliable serum biomarkers which reliably characterize EP. Considerable difficulty in determining and identifying biomarkers for EP diagnosis has been attributed to a number of factors such as the high complexity of serum proteomes; a wide protein abundance range spanning more than 10 orders of magnitude; the presence of most clinically useful biomarkers at very low levels; a high patient-to-patient variability; and potential biases due to variations in sample collection and processing.

There remains a need in the art for a reliable early test for diagnosis of EP and other non-viable pregnancies.

### SUMMARY OF THE INVENTION

In one aspect, a method for diagnosing an abnormal early pregnancy in a mammalian subject comprises contacting a biological sample of the subject with a reagent that enables measurement of a gene, gene fragment, gene transcript, nucleic acid expression product, such as messenger ribonucleic acid (mRNA), for human placental lactogen (hPL) and/or for human chorionic gonadotropin (hCG). The levels of expression, ratio of mRNA to protein expression, or pattern of expression of the selected genes, gene fragments, gene transcripts, nucleic acid expression products or protein expression products in the subject's sample are measured in relation to the level, ratio or pattern of expression of the same genes, gene fragments, gene transcripts, nucleic acid expression products or protein expression products in the same biological fluid of a reference or control female mammalian subject having a normal intrauterine pregnancy (IUP). The presence of, absence of, or changes in expression levels, ratios or patterns of the selected genes, gene fragments, gene transcripts, nucleic acid expression products or protein expression products in relation to those of the reference or control correlates with a diagnosis of abnormal pregnancy.

The sample is in one embodiment material blood, plasma or serum. In one embodiment, the abnormal pregnancy is a miscarriage. In another embodiment, the abnormal pregnancy is an ectopic pregnancy.

In another aspect, a method for diagnosing an abnormal early pregnancy in a mammalian subject comprises contacting a biological sample of the subject with a combination of reagents that detect protein or nucleic acid sequences for each of the biomarkers hPL and hCG.

In one embodiment, absolute levels of the hPL and/or hCG mRNA are measured in the sample and a relation to the reference control is determined. In one embodiment, absolute levels of the hPL and/or hCG protein are measured in the sample and a relation determine to the reference control. In another embodiment, the ratio of the levels of the hPL and/or hCG mRNA to the protein levels in the same sample are measured in the sample and a relation determined to the reference control. In still another embodiment, the pattern or signature formed by the levels or ratios of the hPL and/or hCG in the sample is evaluated in relation to the pattern or signature formed by the same measurements of the same biomarkers in the control.

In another aspect, the methods described above further involves contacting the biological sample of the subject with a reagent that enables measurement of a gene, gene fragment, gene transcript or expression product of at least one additional biomarker; and measuring the levels, ratio or pattern of expression of the selected genes, gene fragments, gene transcripts or expression products and the additional biomarker in the subject's sample and measuring or determining a relation of the sample measurement to the ratio or pattern of expression levels of the same genes, gene fragments, gene transcripts or expression products in the same biological fluid of a reference or control female mammalian subject having a normal intrauterine pregnancy (IUP). Various measurements such as described above for hPL and/or hCG are also provided for the additional biomarker, and form part of the diagnostic result.

In another aspect, a diagnostic reagent, panel, or kit for use in diagnosing an abnormal pregnancy in a mammalian subject contains a reagent that enables measurement in a biological sample of an absolute level or ratio of a gene, gene fragment, gene transcript or nucleic acid expression product for human placental lactogen (hPL) and/or human chorionic gonadotropin (hCG), optionally with that of one or more additional biomarkers; or a reagent that enables measurement in a biological sample of an absolute level or ratio of a protein expression product for hPL and/or hCG, optionally with that of one or more additional biomarkers; or a reagent that enables measurement in a biological sample of an absolute level or ratio of a protein expression product for hCG and/or hPL optionally with that of one or more additional biomarkers. In other embodiments, the diagnostic reagent, panel or kit contains various combinations of such reagents.

In one embodiment, each reagent is a nucleic acid sequence that hybridizes individually to each additional gene, gene fragment, gene transcripts or nucleic acid expression products. In another embodiment, each reagent binds a peptide or protein which is the encoded expression product of each additional biomarker, such as an antibody or fragment thereof.

In still another aspect are reagents including the biomarker mRNA sequences, proteins or fragments thereof associated with a detectable label or immobilized on a suitable substrate.

In another aspect, a kit containing multiple reagents for detection of an abnormal pregnancy biomarker signature is provided. In still other embodiments, optional labels, label systems, substrates for immobilization and controls are included in or with the reagent or kit, and used in these diagnostic methods to identify a characteristic change in the level of expression of the one or more gene, gene fragment, gene transcript, nucleic acid or protein expression product indicative of the diagnosis of abnormal pregnancy.

In another aspect, use of the diagnostic reagents described herein in the methods for the diagnosis of abnormal pregnancy is provided.

Other aspects and advantages of these compositions and methods are described further in the following detailed description of the preferred embodiments thereof.

### DETAILED DESCRIPTION OF THE INVENTION

The compositions and methods described herein provide means for early detection of abnormal pregnancies utilizing certain identified biomarkers, which display characteristic expression level, ratio or pattern of expression levels relative to each other in biological fluids of mammalian subjects with abnormal pregnancy, in contrast to the same fluids of subjects with normal intrauterine pregnancies (IUP). These compositions and methods permit diagnosis of abnormal pregnancy in a more accurate and less invasive manner than currently available.

“Abnormal pregnancy” as used herein means a pregnancy that is no longer progressing, particularly an early stage non-progressive, non-viable pregnancy. Thus, one embodiment of an abnormal pregnancy is an ectopic pregnancy (EP). Another embodiment of abnormal pregnancy is a potential miscarriage. Another embodiment of an abnormal pregnancy is a hydadtiform mole pregnancy. “Patient” or “subject” as used in the methods and compositions described herein means a female mammalian animal, particularly a human. However, the same methods and compositions may be applied in other mammalian subjects, such as a veterinary or farm animal, a domestic animal or pet, and animals normally used for clinical research.

### I. METHODS

In one embodiment, the methods involve the detection and measurement in a sample of maternal biological fluid, e.g., blood, of the expression levels, or ratios, patterns or biomarker signatures formed by expression levels of a gene, gene fragment, gene transcript, nucleic acid expression product, e.g., messenger ribonucleic acid (mRNA), of certain “target” biomarkers. Maternal blood is in direct contact with the syncytiotrophoblast during pregnancy; this tissue constantly undergoes apoptosis and releases microparticles containing RNA and DNA into the maternal blood. Similarly, invasive extravillous trophoblast (EVTB) undergoes apoptosis and may enter the maternal circulation. The inventors theorize that the cellular environment encountered by EVTB in ectopic pregnancy and other abnormal pregnancy differs from that of the normal placenta in that there is no decidua. Moreover, EVTB in ectopic pregnancy may undergo altered cell to cell interaction at the maternal—fetal interface, which influences the rate at which placental RNA enters the maternal circulation.

In these methods described herein, the term “biological fluid” or “sample” are used to refer to any biological fluid or tissue that contains the biomarkers hPL and/or hCG with optional addition biomarkers. In one embodiment, the samples contain biomarker mRNA. In another embodiment, the samples contain biomarker protein. In another embodiment, the samples contain both mRNA and protein forms of biomarkers. As exemplified below, in one embodiment, the samples for use in the methods and with the compositions are blood samples, including maternal blood. In one embodiment, the sample is maternal serum. In another embodiment, the sample is maternal plasma. In another embodiment, the sample is maternal whole blood. In one embodiment, the sample is maternal peripheral blood. However, other maternal biological fluids may also be useful in the methods described herein. In another embodiment, another biological fluid, such as urine, is useful as a sample in these methods. In another embodiment, another biological fluid, such as saliva, is useful as a sample in these methods. In yet further embodiment, vaginal or cervical secretions, amniotic fluid, and placental fluid may be useful. In certain embodiments of the methods described herein, such samples are used without for assay without further pre-treatment. In other embodiments, such samples may further be diluted with saline, buffer or a physiologically acceptable diluent. Alternatively, such samples are concentrated by conventional means.

As used herein, the “target biomarker” includes placental lactogen, preferably human placental lactogen (hPL; also known as chorionic somatomammotropin hormone 1 CSH1), and chorionic gonadotropin, preferably human chorionic gonadotropin (hCG; also known as choriogonadotropin subunit beta precursor CGB), as well as a variety of “additional biomarkers” used in these methods.

The nucleic acid sequence and amino acid sequence for hPL (also known as chorionic somatomammotropin hormone 1 (CSH1)) are publically available, see, e.g., NCBI Database No. NG_{—}028354.1 for the 8754 nucleic acid sequence of hPL and NCBI Database No. P01243.2 for the 217 amino acid sequence of hPL. Other variant sequences for hPL are known and may be used similarly in these methods. It should be understood that, depending upon the context, any reference to hPL also refers to its variants. It should also be understood throughout this specification, that where the subject is not a human, the appropriate analog biomarker or reagent capable of detecting/measuring same would be used.

The nucleic acid sequence and amino acid sequence for hCG are publically available, e.g., NCBI Database No. NM_{—}000737.3 for the nucleic acid sequence of hCG beta subunit (also known as CBG) and NCBI Database No. NP_{—}000728.1 for the amino acid sequence of hCG beta subunit. Other variants are known and may be used similarly in these methods. Fragments of hCG include choriogonadotropin subunit beta precursor (CGB) and glycoprotein hormone alpha chain precursor (CGA). The nucleotide and amino acid sequences for CGA are publically available, see, e.g., NCBI Database Nos. NM_{—}001252383.1 and P01215.1, respectively. It should be understood that, depending upon the context, any reference to hCG also refers to its variants or fragments. It should also be understood throughout this specification, that where the subject is not a human, the appropriate analog biomarker or reagent capable of detecting and/or measuring same would be used.

The term “additional biomarkers” as used herein includes biomarkers, either the genes gene fragments, nucleic acid (mRNA) expression products thereof or protein biomarkers known as pregnancy-related biomarkers from publications cited herein, and including the co-inventors own disclosures, such as Beer et al, “Systematic discovery of ectopic pregnancy serum biomarkers using 3-D protein profiling coupled with label-free quantitation, J. Proteome Research, 10:1126-38 (epub Dec. 10, 2010) (March 2011), including supplemental published materials and methods. In one embodiment, the additional biomarkers include a biomarker of trophoblast function, such as activin A, pregnancy-specific beta 1-glocoprotein (SP1), pregnancy-associated plasma protein A (PAPP-A), or inhibin A. In another embodiment, the additional biomarker is a marker of endometrial function, e.g., glycodelin. In another embodiment, the additional biomarker is a biomarker of angiogenesis, e.g., VEGF. In another embodiment, the biomarker of corpus luteum function is progesterone or inhibin A. In still another embodiment, the additional biomarker is glycoprotein hormone alpha chain precursor (CGA), or progestagen-associated endometrial protein (PAEP). In still another embodiment, still further additional biomarkers can include the pro-domain or extracellular (EC) domain of ADAM12. In still another embodiment, the additional biomarker is Isthmin2 (ISM2). In still another embodiment, the additional biomarker is pregnancy specific beta-1 glycoprotein isoform 1 (PSG1), pregnancy specific beta-1 glycoprotein isoform 7 (PSG7), pregnancy specific beta-1 glycoprotein isoform 11 (PSG11), pregnancy specific beta-1 glycoprotein isoform 9 (PSG9), pregnancy specific beta-1 glycoprotein isoform 2 (PSG2). It should also be understood throughout this specification, that where the subject is not a human, the appropriate analog biomarker or reagent capable of detecting and/or measuring same would be used.

The nucleic acid sequence and amino acid sequences for the above-listed additional biomarkers and desirable fragments are publically available from one or more sources, see, e.g., NCBI Database, GENBANK and Beer et al, cited above, incorporated by reference herein. Fragments of such additional biomarkers may be useful as targets in the methods and compositions described therein. It should be understood that, depending upon the context, any reference to an individual biomarker also refers to such fragments thereof.

The biomarkers identified herein and reagent capable of identifying and measuring same in samples are publically available or readily able to be generated by one of skill in the art. One skilled in the art may readily reproduce the compositions and methods described herein by use of the sequences of the biomarkers, all of which are publicly available from conventional sources, such as NCBI Database or GENBANK.

The “target biomarker signature” is formed by biomarker mRNA expression levels, biomarker proteins/peptides levels, or ratios formed by the relationship of the mRNA to protein or protein to mRNA levels which form a consistent pattern that changes (either in an individual biomarker up-regulated or down-regulated manner) characteristically in the presence of an abnormal pregnancy from that in an IUP. In one embodiment, at least one target biomarker (e.g., hPL or hCG mRNA) forms a suitable biomarker signature for use in the methods and compositions. In one embodiment, at least two target biomarkers (e.g., hPL mRNA and/or hCG mRNA, and an optional biomarker mRNA) form a suitable biomarker signature for use in the methods and compositions. In another embodiment, at least three biomarkers form a suitable biomarker signature for use in the methods and compositions, e.g., hPL mRNA, hCG mRNA and an additional biomarker mRNA. Specific biomarker signatures can include any combination of biomarkers employing at least one biomarker from hPL and hCG and optionally other “additional biomarkers” identified herein. In still further embodiments, at least 5, at least 10, at least 15, at least 20, or more of the additional biomarkers identified herein may form a suitable biomarker signature for the diagnosis of an abnormal pregnancy, such as EP. Specific biomarker signatures can include any combination of biomarkers employing at least one biomarker from hPL and hCG and optionally other “additional biomarkers” identified herein. In still another embodiment, the additional biomarker is a combination of one, 5, or 10 or more of the above identified markers and forms a signature with hPL and/or hCG in the methods described herein.

Thus, in one embodiment, a method for diagnosing an abnormal early pregnancy in a mammalian subject comprises contacting a biological sample of said subject with a reagent that enables detection and/or measurement of a gene, gene fragment, gene transcript, nucleic acid expression product or protein expression product for placental lactogen, preferably human placental lactogen (hPL), a reagent that enables measurement of a gene, gene fragment, gene transcript, nucleic acid expression product or protein expression product for chorionic gonadotropin, preferably human chorionic gonadotropin (hCG), or a combination of reagents for each of hPL and hCG. The levels of expression (e.g., absolute levels or relative levels), the ratio of mRNA to protein expression, or a pattern of expression or signature of the selected genes, gene fragments, gene transcripts, nucleic acid expression products or protein expression products in the subject's sample are evaluated in relation to the levels, ratio or pattern of expression levels of the same genes, gene fragments, gene transcripts, nucleic acid expression products or protein expression products in the same biological fluid of a reference or control female mammalian subject having a normal intrauterine pregnancy (IUP). The presence of, absence of, or changes in expression levels, ratios or patterns of the selected genes, gene fragments, gene transcripts, nucleic acid expression products or protein expression products from those of the reference or control correlates with a diagnosis of abnormal pregnancy.

In such methods the terms “control”, “reference”, “control subject” or “reference subject” are used interchangeably and refer to both an individual female with IUP or the pooled biological fluids (e.g., sera) from multiple females with IUP or to numerical or graphical averages of the expression levels, ratios or patterns of expression/signatures of the target or additional biomarkers obtained from large groups of females with IUP. Such controls are the types that are commonly used in similar diagnostic assays for other biomarkers. Selection of the particular class of controls depends upon the use to which the diagnostic methods and compositions are to be put by the physician. As used herein, the term “predetermined control” refers to a numerical level, average, mean or average range of the expression level or ratio of a biomarker in a defined population or a pattern of multiple levels/ratios for multiple biomarkers. The predetermined control level is preferably provided by using the same assay technique as is used for measurement of the subject's biomarker levels, to avoid any error in standardization. For example, the control may comprise a single healthy pregnant mammalian subject at the same time of pregnancy as the subject. In another embodiment, the control comprises a population of multiple healthy pregnant mammalian subjects at the same time of pregnancy as the subject or multiple healthy IUP mammalian subjects. In another embodiment, the control comprises the same subject at an earlier time or different stage in the pregnancy. In yet another embodiment, the control comprises one or multiple subjects with one or more clinical indicators of abnormal pregnancy, e.g., EP, but who did not develop EP or miscarriage. In addition, a predetermined control may also be a negative predetermined control. In one embodiment, a negative predetermined control comprises one or multiple subjects who have abnormal pregnancies. This control can refer to a numerical average, mean or average range of the expression level or ratio or pattern/signature of one or more biomarkers, in a defined population, rather than a single subject.

One particular embodiment of the method employs determining the relation between the absolute level of the hPL or hCG messenger ribonucleic acid (mRNA) in the sample and the same selected gene's mRNA of the reference or control. Another embodiment of the method employs determining the relation between the absolute levels of hPL mRNA and hCG mRNA in the sample and the same selected gene's mRNA levels of the reference or control, respectively. Still another embodiment of the method employs evaluating the relation between the ratio of the levels of hPL mRNA to hPL protein or the ratio of hPL protein to hPL mRNA levels in the sample to the same ratio of the reference or control. In another embodiment of the method, the measuring step comprises measuring the ratio of the hCG mRNA to hCG protein or protein to mRNA in the sample in relation to the same ratio of the reference or control. Another embodiment involves measuring the ratio of the hPL mRNA to hPL protein and the ratio of the hCG mRNA to hCG protein in the sample in relation to the same ratios, respectively, of the reference or control.

Still another embodiment of a method described herein comprises measuring the absolute level of the hPL or hCG protein expression product in the sample and determining its relation to the same selected gene's protein expression product level of the reference or control. The method can also involve use of measuring the absolute levels of hPL protein expression product and hCG protein expression product in the sample and determining its relation to the same selected gene's protein expression product levels of the reference or control, respectively. In another embodiment, the method measures the ratio of the hPL mRNA to hPL protein (or protein to mRNA) in the sample in relation to the same ratio of the reference or control.

Other alternative embodiments of the method described herein further comprises contacting the biological sample of said subject with a reagent that enables measurement of a gene, gene fragment, gene transcript or expression product of at least one additional biomarker described above; and measuring the levels, ratio or pattern of expression of the selected genes, gene fragments, gene transcripts or expression products and the additional biomarker in the subject's sample and determining a relation to the ratio or pattern of expression levels of the same genes, gene fragments, gene transcripts or expression products in the same biological fluid of a reference or control female mammalian subject having a normal intrauterine pregnancy (IUP). Thus the method can generate a biomarker signature including hPL and/or hCG, with at least one of more of the additional biomarkers. One embodiment of such an alternative method provides for measuring the absolute level of the additional biomarker's mRNA in the sample and determining its relation to the same biomarker's mRNA of the reference or control. Another embodiment of the method comprises measuring the ratio of the additional biomarker's mRNA and determining its relation to the additional biomarker protein (or protein to mRNA) expression product in the sample to the same ratio of the reference or control.

Still another embodiment of the method comprises measuring or detecting a pattern or signature formed by the levels or ratios of the selected and additional biomarker genes, gene fragments, gene transcripts or expression products of the sample in relation to the pattern formed by the same measurements of the same selected and additional biomarker genes, gene fragments, gene transcripts or expression products of the reference or control.

All of the above embodiments of the methods involve subsequent observation and interpretation of a change in expression level of each said selected or additional biomarker gene, gene fragment, gene transcript or expression product to permit diagnosis of abnormal pregnancy or IUP. By “change in expression” is meant an upregulation or downregulation of the genes or mRNA or transcript encoding the biomarkers individually in the sample relative to the reference or control. Such a change can also include an increased or decreased expression level of a selected protein biomarker individually in the sample relative to the reference or control. Such change also includes the ratio formed by the association of an upregulated/downregulated biomarker mRNA with an increased/decreased expression of the biomarker protein in the sample. The change in expression also include a change in the pattern or biomarker signature of expression levels or above ratios formed by the use of multiple biomarkers in the sample in contrast to the pattern or biomarker signature formed by the same biomarker levels or ratios in the reference or control. The degree of change in target expression can vary with each individual and is subject to variation with each population and days or weeks of the pregnancy. For example, in one embodiment, a large change, e.g., 2-3 fold increase or decrease in a small number of biomarkers, e.g., from 1 to 3 characteristic biomarkers, is statistically significant. In another embodiment, a smaller relative change in about 5, 10, 20 or more biomarkers is statistically significant. For example, in one embodiment, a reduced or absent expression level of hPL and/or hCG in the subject's sample is indicative of ectopic pregnancy, as demonstrated by the examples below.

In one embodiment of this method, the detection of hPL and/or hCG mRNA in maternal plasma formed a significant signature that could distinguish between normal intrauterine pregnancy from ectopic pregnancy. In one embodiment, the compositions and methods allow the detection and measurement of the expression levels of the “target” biomarkers hPL and/or hCG mRNA in biological fluid. In another embodiment, the compositions and methods couple the detection and measurement of the expression levels of one or more “target” biomarker hPL and/or hCG peptides or proteins in biological fluids. In another embodiment, the compositions and methods allow the detection and measurement of the ratios of the expression levels of one or more “target” biomarker hPL and/or hCG mRNA to the respective protein expression level in biological fluids.

As described in the Examples below, the inventors determined that hPL mRNA and β-hCG mRNA were undetectable in maternal plasma in most patients with ectopic pregnancy, whereas they could be isolated from most patients with intrauterine pregnancy. Measuring for placental mRNA in maternal plasma is a useful tool for distinguishing normal intrauterine pregnancy from ectopic pregnancy.

In still other embodiments, the methods described herein can include various combinations of these target biomarkers, additional biomarkers, and/or fragments thereof. In another embodiment target mRNA biomarker signatures for use herein include hPL alone, hCG alone, or hPL and hCG. In another embodiment, target mRNA biomarker signature include hPL and activin A, hCG and activin A, or hPL, hCG and activin A and optionally other additional biomarkers. In another embodiment, target mRNA biomarker signature include hPL and SP1, hCG and SP1, or hPL, hCG and SP1 and optionally other additional biomarkers. In another embodiment, target mRNA biomarker signature include hPL and glycodelin, hCG and glycodelin, or hPL, hCG and glycodelin and optionally other additional biomarkers. In another embodiment, target mRNA biomarker signature include hPL and progesterone, hCG and progesterone, or hPL, hCG and progesterone and optionally other additional biomarkers. In another embodiment, target mRNA biomarker signature include hPL and VEGF, hCG and VEGF, or hPL, hCG and VEGF and optionally other additional biomarkers. In another embodiment, target mRNA biomarker signature include hPL and CGA, hCG and CGA, or hPL, hCG and CGA and optionally other additional biomarkers. In another embodiment, target mRNA biomarker signature include hPL and PAPPA, hCG and PAPPA, or hPL, hCG and PAPPA and optionally other additional biomarkers. In another embodiment, target mRNA biomarker signature include hPL and inhibin A, hCG and inhibin A, or hPL, hCG and inhibin A and optionally other additional biomarkers. In another embodiment, target mRNA biomarker signature include hPL and PAEP, hCG and PAEP, or hPL, hCG and PAEP and optionally other additional biomarkers.

In another embodiment, a target mRNA biomarker signature includes hPL and ADAM12, hCG and ADAM12, or hPL, hCG and ADAM12. In yet a further embodiment, a variety of target biomarker mRNA signatures for abnormal pregnancy include combinations of the target biomarkers hPL and/or hCG with two or more of the additional biomarkers identified above, including ISM2, PSG1, PSG7, PSG11, PSG9, PSG2.

In another embodiment, a target mRNA biomarker signature includes hPL and/or hCG, ADAM12, and at least one marker from inhibin A, activin A, VEGF, progesterone, glycodelin, or SP1. In yet a further embodiment, a variety of target biomarker mRNA signatures for abnormal pregnancy include combinations of the target biomarkers hPL and/or hCG with at least one marker from inhibin A, activin A, VEGF, progesterone, glycodelin, or SP1 and one or more of the additional biomarkers identified above, including ISM2, PSG1, PSG7, PSG11, PSG9, PSG2.

Still other additional biomarkers can be obtained for combination with the biomarkers specifically identified here from the documents cited and incorporated herein by reference.

In performing the methods described herein, one of skill in the art may employ conventional nucleic acid and/or protein assays formats which are now conventional in the art.

For example, conventional nucleic acid assays known in the art can be employed for detecting and measuring the gene, gene fragments, gene transcripts and nucleic acid expression products, e.g., mRNA, of the biomarkers in the samples to generate the levels, ratios and patterns of biomarker signature described herein. Such methods include assays based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. See, for example, the procedures described in the example below employing these techniques and reagents for conducting same from commercial suppliers such as Qiagen, Valencia Calif., USA and Quanta Biosciences, Gaithersburg, Md., USA. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

Similarly, for the detection and measurement of biomarker proteins, e.g., for the determination of mRNA to protein ratios, a variety of protein assay formats may be employed in these methods. Among exemplary assay formats are immunoassays using antibodies or ligands to the above-identified biomarkers and biomarker signatures, such as enzyme-linked immunoassays, sandwich immunoassays, homogeneous assays, immunohistochemistry formats, high pressure liquid chromatography (HPLC), multiple reaction monitoring (MRM) mass spectrometry (MS) assays etc. A platform most likely to be used in clinical assays include multi-plexed or parallel sandwich ELISA assays or their equivalent, primarily because this platform is the technology most commonly used to quantify blood proteins in clinical laboratories.

Thus, selection and/or generation of suitable assays for use in the methods described herein are within the skill of the art, provided with this specification, the documents incorporated herein.

### II. DIAGNOSTIC REAGENTS AND KITS

Diagnostic reagents that can detect and measure these targets and methods for evaluating the level, ratio or pattern formed by the levels and ratios of these targets vs. the same measurement of the same biomarkers in normal IUP are valuable tools in the early detection of abnormal pregnancy. Such reagents are selected for detecting and/or measuring the gene, gene fragment, gene transcript or nucleic acid expression product of a target biomarker or additional biomarker. In other embodiments, such reagents include those useful for detecting and/or measuring the protein biomarkers in the biological fluid of the subject.

Thus, a diagnostic reagent, panel, or kit based on the methods described herein can be used in diagnosing an abnormal pregnancy in a mammalian subject. In one embodiment, the reagent, which is present in a kit or on a panel, includes a reagent that enables measurement in a biological sample of an absolute level or ratio of a gene, gene fragment, gene transcript or nucleic acid expression product for human placental lactogen (hPL). In one embodiment, the reagent, which is present in a kit or on a panel, includes a reagent that enables measurement in a biological sample of an absolute level or ratio of a protein expression product for hPL. In one embodiment, the reagent, which is present in a kit or on a panel, includes a reagent that enables measurement in a biological sample of an absolute level or ratio of a gene, gene fragment, gene transcript or nucleic acid expression product for human chorionic gonadotropin (hCG). In one embodiment, the reagent, which is present in a kit or on a panel, includes a reagent that enables measurement in a biological sample of an absolute level or ratio of a protein expression product for hCG.

In another embodiment, the reagent, which is present in a kit or on a panel, includes a reagent that enables measurement in a biological sample of an absolute level or ratio of a gene, gene fragment, gene transcript or nucleic acid expression product of one or more additional biomarker identified herein. In another embodiment, the reagent, which is present in a kit or on a panel, includes a reagent that enables measurement in a biological sample of an absolute level or ratio of a protein expression product for the one or more of the additional biomarkers described herein.

In still a further embodiment, a kit or a panel useful in these methods includes both a reagent that enables measurement in a biological sample of an absolute level or ratio of a gene, gene fragment, gene transcript or nucleic acid expression product for hPL and a reagent that enables measurement in a biological sample of an absolute level or ratio of a protein expression product for hPL. This kit would permit one to generate the nucleic acid expression product levels (absolute or relative), the protein expression product levels (absolute or relative) and a ratio of the hPL mRNA to protein or protein to mRNA.

In still a further embodiment, a kit or a panel useful in these methods includes both a reagent that enables measurement in a biological sample of an absolute level or ratio of a gene, gene fragment, gene transcript or nucleic acid expression product for hCG and a reagent that enables measurement in a biological sample of an absolute level or ratio of a protein expression product for hCG. This kit would permit one to generate the nucleic acid expression product levels (absolute or relative), the protein expression product levels (absolute or relative) and a ratio of the hCG mRNA to protein or protein to mRNA.

In still a further embodiment, a kit or a panel useful in these methods includes a combination of at least two of a reagent that enables measurement in a biological sample of an absolute level or ratio of a gene, gene fragment, gene transcript or nucleic acid expression product for hPL, a reagent that enables measurement in a biological sample of an absolute level or ratio of a gene, gene fragment, gene transcript or nucleic acid expression product for hCG, and a reagent that enables measurement in a biological sample of an absolute level or ratio of a gene, gene fragment, gene transcript or nucleic acid expression product of one or more additional biomarker identified herein.

In still a further embodiment, a kit or a panel useful in these methods includes a combination of at least two of a reagent that enables measurement in a biological sample of an absolute level or ratio of a protein expression product for hPL, a reagent that enables measurement in a biological sample of an absolute level or ratio of a protein expression product for hCG and a reagent that enables measurement in a biological sample of an absolute level or ratio of a protein expression product for the one or more of the additional biomarkers described herein.

In still a further embodiment, a kit or a panel useful in these methods includes a combination of at least two of a reagent that enables measurement in a biological sample of an absolute level or ratio of a gene, gene fragment, gene transcript or nucleic acid expression product for hPL, a reagent that enables measurement in a biological sample of an absolute level or ratio of a protein expression product for hPL, a reagent that enables measurement in a biological sample of an absolute level or ratio of a gene, gene fragment, gene transcript or nucleic acid expression product for hCG, and a reagent that enables measurement in a biological sample of an absolute level or ratio of a protein expression product for hCG.

In still a further embodiment, a kit or a panel useful in these methods includes a combination of at least two of a reagent that enables measurement in a biological sample of an absolute level or ratio of a gene, gene fragment, gene transcript or nucleic acid expression product for hPL, a reagent that enables measurement in a biological sample of an absolute level or ratio of a protein expression product for hPL, a reagent that enables measurement in a biological sample of an absolute level or ratio of a gene, gene fragment, gene transcript or nucleic acid expression product of one or more additional biomarker identified herein, and a reagent that enables measurement in a biological sample of an absolute level or ratio of a protein expression product for the one or more of the additional biomarkers described herein.

In still a further embodiment, a kit or a panel useful in these methods includes a combination of at least two of a reagent that enables measurement in a biological sample of an absolute level or ratio of a gene, gene fragment, gene transcript or nucleic acid expression product for hCG, a reagent that enables measurement in a biological sample of an absolute level or ratio of a protein expression product for hCG, a reagent that enables measurement in a biological sample of an absolute level or ratio of a gene, gene fragment, gene transcript or nucleic acid expression product of one or more additional biomarker identified herein, and a reagent that enables measurement in a biological sample of an absolute level or ratio of a protein expression product for the one or more of the additional biomarkers described herein. In still other embodiments, the methods described herein can include various combinations of these target biomarkers, additional biomarkers, and/or fragments thereof.

For example, in one embodiment, a kit contains reagents for the measurement and detection of hPL alone, hCG alone, or hPL and hCG. In another embodiment a kit contains reagents for the measurement and detection of hPL and activin A, hCG and activin A, or hPL, hCG and activin A and optionally other additional biomarkers. In another embodiment, a kit contains reagents for the measurement and detection of hPL and SP1, hCG and SP1, or hPL, hCG and SP1 and optionally other additional biomarkers. In another embodiment, a kit contains reagents for the measurement and detection of hPL and glycodelin, hCG and glycodelin, or hPL, hCG and glycodelin and optionally other additional biomarkers. In another embodiment, a kit contains reagents for the measurement and detection of hPL and progesterone, hCG and progesterone, or hPL, hCG and progesterone and optionally other additional biomarkers. In another embodiment, a kit contains reagents for the measurement and detection of hPL and VEGF, hCG and VEGF, or hPL, hCG and VEGF and optionally other additional biomarkers. In another embodiment, a kit contains reagents for the measurement and detection of hPL and CGA, hCG and CGA, or hPL, hCG and CGA and optionally other additional biomarkers. In another embodiment, a kit contains reagents for the measurement and detection of hPL and PAPPA, hCG and PAPPA, or hPL, hCG and PAPPA and optionally other additional biomarkers. In another embodiment, a kit contains reagents for the measurement and detection of hPL and inhibin A, hCG and inhibin A, or hPL, hCG and inhibin A and optionally other additional biomarkers. In another embodiment, a kit contains reagents for the measurement and detection of hPL and PAEP, hCG and PAEP, or hPL, hCG and PAEP and optionally other additional biomarkers.

In another embodiment, a kit contains reagents for the measurement and detection of hPL and ADAM12, hCG and ADAM12, or hPL, hCG and ADAM12. In another embodiment, a kit contains reagents for the measurement and detection of a variety of target biomarkers including hPL and/or hCG with reagents for two or more of the additional biomarkers identified above, including ISM2, PSG1, PSG7, PSG11, PSG9, PSG2.

In another embodiment, a kit contains reagents for the measurement and detection of hPL and/or hCG, ADAM12, and at least one marker from inhibin A, activin A, VEGF, progesterone, glycodelin, or SP1. In another embodiment, a kit contains reagents for the measurement and detection of a variety of target biomarkers hPL and/or hCG with at least one marker from inhibin A, activin A, VEGF, progesterone, glycodelin, or SP1 and one or more of the additional biomarkers identified above, including ISM2, PSG1, PSG7, PSG11, PSG9, PSG2.

Still other reagents as described above for additional biomarkers can be obtained for combination with the biomarkers specifically identified here from the documents cited and incorporated herein by reference.

In yet another kit or panel can comprise a combination of all of the above noted reagents. The biomarker sequences themselves may also be useful as reagents. A kit for diagnosing abnormal pregnancy, e.g., EP, in a mammalian subject as described herein can contain multiple reagents or one or more individual reagents.

In certain embodiments, at least one reagent can be associated with a detectable label and/or immobilized on a substrate. For these reagents, the labels may be selected from among many known diagnostic labels, including those described herein. Similarly, the substrates for immobilization may be any of the common substrates, such as glass or plastic. For example, one embodiment of a reagent includes a substrate upon which the biomarker sequences themselves, polynucleotides or oligonucleotides, or ligands are immobilized. In another embodiment, the kit also contains optional detectable labels, immobilization substrates, optional substrates for enzymatic labels, as well as other laboratory items. The reagents described herein, optionally associated with detectable labels, can be presented in the format of a microfluidics card, a chip or chamber, a microarray or a kit adapted for use with the assays described in the examples or below, e.g., ELISAs or PCR, RT-PCR or Q PCR techniques described herein. The term “microarray” refers to an ordered arrangement of hybridizable array elements, e.g., primers, probes, ligands, biomarker nucleic acid sequence or protein sequences on a substrate.

As used herein, “labels” or “reporter molecules” are chemical or biochemical moieties useful for labeling a nucleic acid (including a single nucleotide), polynucleotide, oligonucleotide, or protein ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to an oligonucleotide or nucleotide (e.g., a non-natural nucleotide) or ligand. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength. Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to reagents useful in diagnostic assays of this invention may be easily selected from among numerous compositions known and readily available to one skilled in the art of diagnostic assays. The reagents useful in this invention are not limited by the particular detectable label or label system employed.

Among suitable reagents for detecting and measuring the nucleic acid sequences (e.g., mRNA of the biomarkers) are labeled or immobilized polynucleotides or oligonucleotides that hybridize to genes, gene fragments, gene transcripts or other nucleic acid expression products of the target or additional biomarkers useful in these methods. For example, in one embodiment, the diagnostic reagent is a polynucleotide or oligonucleotide sequence that hybridizes to gene, gene fragment, gene transcript or nucleotide sequence, e.g., mRNA of hPL, hCG and optionally one or more of the additional biomarkers, or a unique fragment thereof.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule.

By “hybridize” is meant pair to form a double-stranded molecule between complementary polynucleotide sequences (e.g., a biomarker gene described herein), or portions thereof, under various conditions of stringency. For example, stringent salt concentration can be less than about 750 mM NaCl and 75 mM trisodium citrate. In one embodiment, the stringency conditions are less than about 500 mM NaCl and 50 mM trisodium citrate or less than about 250 mM NaCl and 25 mM trisodium citrate. Low stringency hybridization can be obtained in the absence of organic solvent. High stringency hybridization involves the presence of organic solvent, for example, about 35% or greater formamide. Suitable temperatures for stringent hybridization include temperatures from about 30° C. to about 42° C. Other hybridization conditions, e.g., time of hybridization reaction, detergent presence and concentration, etc., are readily known and readily selected by one of skill in the art. Hybridization techniques are described in conventional texts and publications, such as Wahl, G. M. and S. L. Berger, Methods Enzymol. 152:399 (1987); Ausubel et al. (eds), Current Protocols in Molecular Biology, Wiley Interscience, New York (2001); and Sambrook et al., Molecular Cloning: A Laboratory Manual, Cold Spring Harbor Laboratory Press, New York (2001) among others.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Any combination of labeled or immobilized biomarker-hybridizable sequences can be assembled in a diagnostic kit for the purposes of diagnosing abnormal pregnancy. In one embodiment, these polynucleotide or oligonucleotide reagent(s) are part of a primer-probe set, and the kit comprises both primer and probe. Each the primer-probe set amplifies a different gene, gene fragment or gene expression product that encodes a different target or additional biomarker. In another aspect, suitable embodiments of such labeled or immobilized reagents include at least one, 2, 3, 4, 5, 6, 7 or 8 polynucleotide/oligonucleotides. Each polynucleotide/oligonucleotide hybridizes to a gene, gene fragment, gene transcript or nucleic acid expression product of a single target biomarker, additional biomarker, or fragments thereof. The reagent, panel or kit, can be or contain a nucleic acid primer or probe that hybridizes to the gene, gene fragment, gene transcript or nucleic acid expression product of hPL, hCG or an optional additional biomarker.

PCR primers are oligonucleotides which act as points of initiation of synthesis when placed under suitable conditions in which synthesis of a primer extension product which is complementary to a nucleic acid strand is induced, and are generated using techniques known to those of skill in the art (see the texts cited above). Each of the PCR primer sets is composed of a 5′ primer and a 3′ primer, preferably single stranded. Double stranded primers can be used, but are generally treated to separate the strands before extension products are prepared. The primers may be about 15 to 25 or more nucleotides, and preferably at least 18 nucleotides. However, for certain applications shorter nucleotides, e.g., 7 to 15 nucleotides are utilized. See, for example, the primers of Table 1 below.

The primers are sufficiently complementary to the respective strand of the target sequence to be amplified, so that each primer hybridizes with its respective strand. The primer sequence need not be a 100% complementary match for sequence being amplified. For example, a non-complementary nucleotide sequence may be attached to the 5′ end of the primer, with the remainder of the primer sequence being completely complementary to the strand. Alternatively, non-complementary bases can be interspersed into the primer. The primer has sufficient complementarity with the sequence of the strand to be amplified to hybridize therewith and form a template for synthesis of the extension product of the other primer. The PCR primers and probes are designed based upon suitable intron sequences present in the biomarker gene(s). The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in publications such as U.S. Pat. No. 7,081,340. See also the available tools and manufacturers' instructions for tools including DNA BLAST software, the Repeat Masker program (Baylor College of Medicine), Primer Express (Applied Biosystems); MGB assay-by-design (Applied Biosystems), etc. In general, optimal PCR primers and probes are generally 17-30 bases in length, and contain about 20-80%, such as, for example, about 50-60% G+C bases. Melting temperatures of between 50 and 80° C., e.g. about 50 to 70° C. are typically preferred. See, e.g., Table 1.

Among other suitable reagents for use in the methods described above are labeled or immobilized biomarker nucleic acid or peptide sequences. In one embodiment, a diagnostic reagent for use in the methods of diagnosing abnormal pregnancy includes a target biomarker and optionally an additional biomarker identified herein, associated with a detectable label or portion of a detectable label system or immobilized on a substrate. In still another embodiment, combinations of such labeled or immobilized biomarker sequences are suitable reagents and components of a diagnostic kit.

In another aspect, suitable embodiments of such labeled or immobilized reagents include at least one, 2, 3, 4, 5, 10, 20 or more of the target biomarkers (nucleic acid sequences and/or protein sequences) and optional additional biomarkers or their fragments thereof. Still other diagnostic reagents are surrogate peptides used for MRM assays.

Any combination of labeled or immobilized biomarkers can be assembled in a diagnostic kit for the purposes of diagnosing an abnormal pregnancy, such as EP, including all of the combinations identified above.

Among other suitable reagents for use in the methods described above are labeled or immobilized ligands that bind the biomarkers in protein form. The term “ligand” refers to a molecule that binds to a protein or peptide, and includes antibodies and fragments thereof. As used herein, the term “antibody,” refers to an immunoglobulin molecule which is able to specifically bind to a specific amino acid sequence or antigen. Antibodies useful in the method to identify protein biomarkers include, for example, polyclonal antibodies, monoclonal antibodies, intracellular antibodies (“intrabodies”), diabodies, recombinant antibodies, chimeric antibodies, Fv, Fab and F(ab)₂ fragments, as well as single chain antibodies (scFv), camelid antibodies and humanized antibodies (See e.g., Harlow et al., 1999, Using Antibodies: A Laboratory Manual, Cold Spring Harbor Laboratory Press, NY). Fragments and components (e.g., CDRs, single chain variable regions, etc.) may be used in place of antibodies. Such antibodies may be presently extant in the art or presently used commercially, such as those available as part of commercial antibody ELISA assay kits or that may be developed by techniques now common in the field of immunology.

A recombinant molecule bearing the binding portion of an EP biomarker antibody, e.g., carrying one or more variable chain CDR sequences that bind e.g., hPL, may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same EP biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers or a particular sequence of the selected EP biomarker may be employed in the methods of the present invention, regardless of how the antibody or mixture of antibodies was generated.

Similarly, the antibodies may be tagged or labeled with reagents capable of providing a detectable signal, depending upon the assay format employed. Such labels are described above. Where more than one antibody is employed in a diagnostic method, e.g., such as in a sandwich ELISA, the labels are desirably interactive to produce a detectable signal.

Other ligands known to bind amino acid sequences may be useful in these methods. Such a ligand desirably binds to a target or additional biomarker or a peptide contained therein that appears in the biological fluid of the subject and can be employed to generate the ratios and/or patterns of biomarkers/biomarker signatures along with the reagents that detect and/or measure nucleic acid sequences characteristic of the biomarkers, e.g., mRNA measurements.

The ligand itself may be labeled or immobilized. In another aspect, suitable embodiments of such labeled or immobilized reagents include at least one, 2, 3, 4, 5, 10 or more such ligands. Each ligand binds to a single biomarker or fragment thereof. Thus, the reagent, panel or kit, can be or comprise is a ligand that binds to the protein hPL, hCG or the additional biomarker.

In yet further embodiments, other reagents for the detection of protein biomarkers in biological samples, such as peptide mimetics, synthetic chemical compounds capable of detecting the selected biomarker may be used in other assay formats for the quantitative detection of biomarker protein in biological samples.

The selection of the ligands, poly/oligonucleotide sequences, their length, suitable labels and substrates used in the composition are routine determinations made by one of skill in the art in view of the teachings of which biomarkers form signature suitable for the diagnosis of abnormal pregnancy.

These diagnostic methods and reagents allow for identifying abnormal pregnancy at an earlier stage than is currently possible with today's protocols. Identification of abnormal pregnancies at an earlier gestational age has a numer of advantages, including saving patients from trauma of undergoing expensive, dangerous surgical procedures needed for treatment of a abnormal pregnancy of later gestation and decreasing the number of women experienceing life-threatening hemoperitoneum. A minimally invasive blood test, or test of other biological fluid, for recognition of changes in expression levels, ratios or patterns of expression levels provides a diagnostic test less subject to interpretation errors, than ultrasound and other technologies.

### III. EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

The following example measure and evaluate placental mRNA expression in the maternal circulation among women with intrauterine and ectopic pregnancies.

### Example 1

### Evaluation of IUP and EP

Women in the first trimester of pregnancy were asked to participate in a pilot study at the University of Miami, Miller School of Medicine Miami, USA, between Oct. 1, 2009, and Aug. 1, 2010. The study was approved by the Institutional Review Boards of the University of Miami; all participants provided written informed consent. Blood samples were obtained from women in early pregnancy. Demographic and clinical data were prospectively entered into a computerized database. Participants were followed until they were definitively diagnosed. A visualized intrauterine pregnancy was defined as an intrauterine pregnancy identified via ultrasound, with a yolk sac or a fetal pole. Ectopic pregnancy was defined as either visualized (extrauterine gestational sac with yolk sac or embryonic cardiac activity identified via ultrasound, or an ectopic visualized at the time of surgery) or nonvisualized (rising hCG level after uterine evacuation) ectopic pregnancy.

Plasma samples from women with normal intrauterine pregnancy or ectopic pregnancy were used for RNA isolation and quantitative reverse-transcription polymerase chain reaction (RT-PCR). The JEG-3 human choriocarcinoma cell line was used as a positive control. Total RNA extraction from 5×10⁶ JEG-3 cells was performed using the RNeasy Mini Kit (Qiagen, Valencia Calif., USA), and total RNA extraction from patient samples using 2 mL of plasma was performed using the QIAMP Circulating Nucleic Acid Kit (Qiagen). Reverse transcription of total RNA to cDNA was performed using the QSCRIPT cDNA Synthesis Kit (Quanta Biosciences, Gaithersburg, Md., USA). cDNA samples were then used to perform quantitative gene expression analysis (qPCR) of hPL and β-hCG using gene-specific TAQMAN primer and probe sets (Biosearch Technologies, Novato, Calif., USA). The gene encoding GAPDH was used as a housekeeping control gene.

Multiplexed qPCR reactions were prepared using PERFETA Multiplex qPCR Super Mix (Quanta Biosciences) reagents with a final concentration of 300-nM forward and reverse primers, and a 200-nM probe for each gene. The thermal profile used for the multiplexed hPL/β-hCG/GAPDH gene expression analysis was as follows: 1 cycle at 95° C. for 3 minutes, followed by 40 cycles at 95° C. for 15 seconds and 60° C. for 1 minute. All gene expression data were collected and analyzed. The primer and probe sequences for each of the genes used are listed in Table 1. The fluorescent labels and quenchers for each amplicon probe are shown at the 5′ and 3′ ends of the sequences, respectively.

Transcript copy numbers of each target gene were quantified via absolute quantification. To establish standard curves for each target gene, serial dilutions were prepared from single-stranded synthetic DNA oligonucleotides (IDT Technologies, San Diego, Calif., USA) corresponding to the amplicons from each target, ranging from 1×10⁹ to 1 copy. Polymerase chain reaction efficiencies between 95% and 105% for each standard curve were deemed acceptable for use of target gene quantification. Raw threshold values for each gene target were used in concordance with the standard curve prepared for respective genes to determine the copy number present in each patient sample. The copy numbers for each target gene were then normalized for relative quantification by dividing the target gene copy number by the housekeeping gene copy number.

Continuous data were evaluated using the Student t test if the distribution of samples was normal; if the sample distribution was asymmetric, the Mann-Whitney U test was used. Pb 0.05 was considered to be statistically significant. All statistical calculations were performed using SIGMASTAT software (SPSS, Chicago, Ill., USA).

TABLE 1
PRIMER AND PROBE SEQUENCES USED FOR QUANTITATE
GENE EXPRESSION ANALYSIS.

| | PRIMER | | |
|---|---|---|---|
| | DIRECTION/ | | SEQ |
| GENE NAME | PROBE | SEQUENCE | ID NOS |
| hPL | Forward | 5′-CATGACTCCCAGACCTCCTTC-3′ | 1 |
| | Reverse | 5′-TGCGGAGCAGCTCTAGATTG-3′ | 2 |
| | Probe | 5′-FAM-TTCTGTTGCGTTTCCTCCATGTTGG-BHQ1-3′ | 3 |
| hCG | Forward | 5′-CTACTGCCCCACCATGACCC-3′ | 4 |
| | Reverse | 5′TGGACTCGAAGCGCACATC-3′ | 5 |
| | Probe | 5′-CAL560-CCTGCCTCAGGTGGTGTGCAACTAC-BHQ1-3′ | 6 |
| GAPDH | Forward | 5′-CCACTCCTCCACCCTTGAC-3′ | 7 |
| | Reverse | 5′-ACCCTGTTGCTGTAGCCA-3′ | 8 |
| | Probe | 5′-CAL610-TTGCCCTCAACGACCACTTTGTC-BHQ2-3′ | 9 |

The results of the experiment were as follows: Of the 25 participants, 12 were diagnosed with ectopic pregnancy, while 13 were found to have viable intrauterine pregnancies. The clinical characteristics of the participants are shown in Table 2. Women in the ectopic group were similar to those in the intrauterine group with regard to age, gravidity, parity, and estimated gestational age based on last menstrual period. Levels of hCG were significantly higher in the intrauterine group than in the ectopic group (35 696±22 000 mIU/mL vs 2704±2981 mIU/mL; P=0.01).

mRNA for hPL could not be detected in 10 of the 12 women with ectopic pregnancy, in contrast to 1 of the 13 participants with intrauterine pregnancy (specificity 92%; sensitivity 83%; positive predictive value, PPV=91%; negative predictive value, NPV=86%). Patients with ectopic pregnancy were 6 times more likely to have undetectable levels of hPL mRNA (relative risk, RR=6.36; 95% confidence interval, CI 1.70-23.20; Pb 0.01).

mRNA for hCG could not be detected in 11 of the 12 participants with ectopic pregnancy, in contrast with 3 of the 13 women with normal intrauterine pregnancy (specificity 77%; sensitivity 92%; PPV=78%; NPV=91%). Patients with ectopic pregnancy were 8 times more likely to have undetectable levels of hCG mRNA (RR=8.64; 95% CI, 1.30-57.10; Pb 0.01).

mRNA copy numbers for both hPL and hCG (normalized by GAPDH) were significantly lower in the ectopic pregnancy group than in the intrauterine pregnancy group (Table 2).

TABLE 2
CLINICAL CHARACTERISTICS AND MRNA LEVELS OF WOMEN
WITH INTRAUTERINE OR ECTOPIC PREGNANCIES^{A}.

| | INTRAUTERINE | ECTOPIC | P |
|---|---|---|---|
| CHARACTERISTIC | GROUP (N = 13) | GROUP (N = 12) | VALUE |
| Age, y | 30 ± 6 | 28 ± 5 | 0.43 |
| Gravidity | 2 (1-6) | 2 (1-10) | 0.78 |
| Parity | 1 (1-3) | 1 (0-8) | 0.57 |
| Gestational age, wk | 7 ± 4 | 7 ± 3 | 0.84 |
| hCG, mIU/mL | 35 696 ± 22 000 | 2704 ± 2981 | 0.01 |
| Relative copy number of | 68 ± 149 | 2 ± 9 | 0.03 |
| hCG mRNA | | | |
| Relative copy number of | 285 ± 651 | 35 ± 285 | 0.05 |
| hPL mRNA | | | |

Abbreviations: hCG, human chorionic gonadotropin; hPL, human placental lactogen.
^{a}Values are given as mean ± SD or median (range) unless otherwise indicated.

Maternal blood is in direct contact with the syncytiotrophoblast (ST) from the normal placenta during pregnancy. This tissue constantly undergoes apoptosis and releases microparticles containing RNA and DNA into the maternal blood. Similarly, invasive extravillous trophoblast (EVTB) undergoes apoptosis and may enter the maternal circulation. The inventors theorize that the cellular environment encountered by EVTB in ectopic pregnancy differs from that of the normal placenta in that there is no decidua. Moreover, EVTB in ectopic pregnancy may undergo altered cell-cell interaction at the maternal-fetal interface. These changes influence the rate at which placental RNA enters the maternal circulation. The following example measures and contrasts placental mRNA expression in the maternal circulation among women with intrauterine and ectopic pregnancies.

The investigators quantitated levels of two mRNAs associated with genes expressed by the placenta hPL and beta-HCG, normalized to the housekeeping gene GAPDH. It was determined that placental mRNA in the maternal circulation is present in significantly lower copies from women with an EP in contrast to IUP. The current study found that patients with ectopic pregnancy were 6 times more likely to have undetectable levels of hPL mRNA and 8 times more likely to have undetectable levels of hCG mRNA. Measurement of placental mRNA in the maternal circulation thus helps to distinguish between intrauterine and ectopic pregnancies.

Although placentation is relatively similar in ectopic and intrauterine pregnancies, there are certain differences such as the absence of decidua formation by the tube and the impossibility of trophoblast formation and differentiation within the tube. The inventors hypothesize that there is a decreased blood supply in ectopic pregnancy, which may affect the transport of EVTB into the maternal circulation, with a subsequent decrease in levels of mRNA in maternal plasma. Furthermore, the decreased blood supply could be caused by not only the lack of space for the trophoblast to develop but also the structural differences of the tubal vessels.

Limitations of the present study were the small sample size and the significant difference in hCG levels between the 2 groups. Moreover, only women with intrauterine or ectopic pregnancies were investigated; women who experienced spontaneous abortion were not included. In addition, previous studies have shown that placental mRNA levels are higher in the cellular component of maternal blood than in the plasma during early pregnancy. In the present study, only the plasma component—not the cellular component, which may behave differently—was analyzed. Evaluation of both cellular and plasma mRNA is predicted to support the example's conclusions. In summary, lower levels of placental mRNA were detected in the maternal blood of women with ectopic pregnancies in contrast to women who had intrauterine pregnancies. The measurement of placental mRNA in the blood of women at risk for ectopic pregnancy is a useful test for distinguishing ectopic from intrauterine pregnancies.

### Example 2

### Evaluation of IUP, EP and SAB

To measure and evaluate cellular placental mRNA expression in the maternal circulation from women with an intrauterine pregnancy (IUP), spontaneous abortion (SAB) or ectopic pregnancy (EP).

Whole blood samples were obtained from twenty-five women with early pregnancies at risk for an EP. Demographics and clinical data were prospectively collected and entered into a computerized database. Women were followed until they were definitively diagnosed: 25 women were diagnosed with an EP, 24 with a SAB and 28 were found to have a viable IUP. Women in the EP group were similar in respect to age, gravidity, parity and estimated gestational age based on the last menstrual period. A visualized IUP was defined as an IUP identified by ultrasound with a yolk sac or a fetal pole. The diagnosis of EP was either a visualized EP (extra uterine gestational sac with yolk sac or embryonic cardiac activity identified with ultrasound or an ectopic visualized at the time of surgery) or a non-visualized EP (defined as a rising hCG level after uterine evacuation).

Whole blood samples from women with normal SAB, IUP or EP were collected in PAXGENE blood RNA tubes. Cellular mRNA was isolated from the maternal plasma and quantitative RT-PCR was performed to measure cellular mRNA for hCG and hPL. GAPDH mRNA expression was used as an internal control. PREANALYTIX, Hombrechtikon, Switzerland, was used for RNA isolation and quantitative RT-PCR. The JEG-3 human choriocarcinoma cell line served as a positive control.

Total RNA extraction from 5×10⁶ JEG-3 cells was performed using the RNEASY Mini Kit (Qiagen Inc, Valencia Calif., USA), and total RNA extraction from patient samples using 2 ml of plasma was performed using the QIAMP Circulating Nucleic Acid kit (Qiagen Inc, Valencia Calif., USA). Reverse transcription of total RNA to cDNA was performed using the QSCRIPT cDNA Synthesis Kit (Quanta Biosciences, Gaithersburg Md., USA). cDNA samples were then used to perform quantitative gene expression analysis (qPCR) of hPL and β-hCG using gene-specific TAQMAN primer and probe sets (Biosearch Technologies, Novato, USA). GAPDH was used as a housekeeping control gene. Multiplexed quantitative PCR reactions were prepared using PERFETA Multiplex qPCR Super Mix reagents (Quanta Biosciences, Gaithersburg Md., USA) with a final concentration of 300 nM forward and reverse primers, and 200 nM probe for each gene. The thermal profile used for the multiplexed hPL/β-hCG/GAPDH gene expression analysis was as follows: 1 cycle at 95° C. for 3 min, followed by 40 cycles at 95° C. for 15 seconds and 60° C. for 1 min. All gene expression data were collected and analyzed. The primer and probe sequences for each of the genes used are listed in Table 1 in Example 1.

Transcript copy numbers of each target gene were quantified by absolute quantification. To establish standard curves for each target gene, serial dilutions were prepared from single-stranded synthetic DNA oligonucleotides (IDT Technologies, San Diego Calif., USA) corresponding to the amplicons from each target, ranging from 1×10⁹ to 1×10⁰ copies. PCR efficiencies between 95% and 105% for each standard curve were deemed acceptable for use of target gene quantification. Raw threshold (C_{T}) values for each gene target were used in concordance with the standard curve prepared for respective genes to determine the copy number present in each patient sample.

Continuous data were evaluated using the Student's t test if the distribution of samples was normal or the Mann-Whitney U test if the sample distribution was asymmetrical. Differences were considered significant when P-value was less than 0.05. All statistical calculations were performed using the SigmaStat software (SPSS Inc, Chicago, Ill.).

Results:

hCG levels were significantly higher in the IUP group in contrast with the EP group and the SAB group [mean±SD, International Units, 24384±31905 vs. 6435±1747 P-value <0.01, vs. 10449±16679 P-value 0.02]. Cellular mRNA for hCG could not be detected in 19 out of 25 women with an EP, 11 out of 24 in SAB and 12 out of 26 with a normal IUP. Patients with an EP were 2 times more likely to have no detectable level of cellular hCG mRNA in contrast to IUP (RR=2.25, 95% CI 1.07-4.70, P<0.03). There was no difference between the SAB and IUP in the rate of non-detectable hCG mRNA. Patients with an EP were 2 times more likely to have no detectable level of cellular hCG mRNA relative to IUP (RR=2.25, 95% CI 1.07-4.70, P<0.03). There was no difference between the SAB and IUP in the rate of non-detectable cellular hCG mRNA.

Cellular mRNA copy numbers both for hPL and hCG were significantly lower in the EP group compared to the IUP group [hCG mRNA (relative copy number) IUP vs. EP, 3.579±5.901 vs. 0.577±1.25, P-value=0.02, hPL mRNA (relative copy number) IUP vs. EP, 11.22±24.26 vs. 6.317±15.38, P-value<0.01]. Cellular mRNA for hPL could not be detected in 9 out of 25 women with EP, 7 out of 24 in SAB and only 6 out of 13 with an IUP. There was no difference between the EP and IUP or SAB and IUP in the rate of non-detectable cellular hPL mRNA. There was no difference between SAB and IUP in cellular mRNA copy numbers both for hPL and hCG.

Thus, placental cellular mRNA in the maternal circulation is present in significantly lower copies from women with an EP in contrast to IUP. Measurement of placental mRNA in the maternal circulation distinguishes between an IUP and EP, but not between an IUP and SAB.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein. As used herein, the term “about” is defined as a variability of 10% from the reference given, unless otherwise specified.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

Each and every patent, patent application, and publication, including the above-noted provisional application, publications listed below, and publically available peptide sequences cited throughout the disclosure, as well as the Sequence Listing, is expressly incorporated herein by reference in its entirety. In addition, Takacs P, et al, Placental mRNA in maternal plasma as a predictor of ectopic pregnancy, Int J Gynecol Obstet (2012), doi:10.1016/j.ijgo.2011.12.011 is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

### PUBLICATIONS

1. Chang J, et al. Pregnancy related mortality surveillance-United States, 1991-1999. MMWR Surveill Summ 2003; 52(2):1-8.
2. Hoover K W et al, Trends in the diagnosis and treatment of ectopic pregnancy in the United States. Obstet Gynecol 2010; 115(3):495-502.
3. Lipscomb G H. Ectopic pregnancy: still cause for concern. Obstet Gynecol 2010; 115:487-8.
4. Barnhart K T. Clinical practice. Ectopic pregnancy. N Engl J Med 2009; 361(4): 379-87.
5. Rausch M E, et al., Development of a multiple marker test for ectopic pregnancy. Obstet Gynecol 2011; 117(3):573-82.
6. Tong Y K, Lo Y M. Diagnostic developments involving cell-free (circulating) nucleic acids. Clin Chim Acta 2006; 363(1-2):187-96.
7. Wataganara T, Bianchi D W. Fetal cell-free nucleic acids in the maternal circulation: new clinical applications. Ann N Y Acad Sci 2004; 1022:90-9.
8. Picchiassi E, et al., Identification of universal mRNA markers for noninvasive prenatal screening of trisomies. Prenat Diagn 2010; 30(8):764-70.
9. Ng E K, et al. Evaluation of human chorionic gonadotropin beta-subunit mRNA concentrations in maternal serum in aneuploid pregnancies: a feasibility study. Clin Chem 2004; 50(6): 1055-7.
10. Masuzaki H, et al. Placental mRNA in maternal plasma and its clinical application to the evaluation of placental status in a pregnant woman with placenta previa-percreta. Clin Chem 2005; 51(5):923-5.
11. Boye K, et al., Novel feto-specific mRNA species suitable for identification of fetal cells from the maternal circulation. Prenat Diagn 2001; 21(10):806-12.
12. Poon L L, et al., Presence of fetal RNA in maternal plasma. Clin Chem 2000; 46(11):1832-4.
13. Tsui N B. Dennis Lo Y M. Placental RNA in maternal plasma: toward noninvasive fetal gene expression profiling. Ann N Y Acad Sci 2006; 1075:96-102.
14. Poon L L, et al, Circulating fetal RNA in maternal plasma. Ann NY Acad Sci 2001; 945:207-10.
15. Ng E K, Tsui N B, Lau T K, Leung T N, Chiu R W, Panesar N S, et al. mRNA of placental origin is readily detectable in maternal plasma. Proc Natl Acad Sci USA 2003; 100 (8):4748-53.
16. Johansen M, Redman C W, Wilkins T, Sargent Ill. Trophoblast deportation in human pregnancy—its relevance for pre-eclampsia. Placenta 1999; 20(7):531-9.
17. Kumpel B, King M J, Sooranna S, Jackson D, Eastlake J, Cheng R, et al. Phenotype and mRNA expression of syncytiotrophoblast microparticles isolated from human placenta. Ann N Y Acad Sci 2008; 1137:144-7.
18. Randall S, Buckley C H, Fox H. Placentation in the fallopian tube. Int J Gynecol Pathol 1987; 6(2):132-9.
19. Okazaki S. et al, Measurement of mRNA of trophoblast-specific genes in cellular and plasma components of maternal blood. J Med Genet 2006; 43(9):e47.
20. Takacs P, et al, Placental mRNA in maternal plasma as a predictor of ectopic pregnancy, Int J Gynecol Obstet (2012), doi:10.1016/j.ijgo.2011.12.011
21. Beer et al, “Systematic discovery of ectopic pregnancy serum biomarkers using 3-D protein profiling coupled with label-free quantitation, J. Proteome Research, 10:1126-38 (epub Dec. 10, 2010) (March 2011)

1. A method for diagnosing an abnormal early pregnancy in a mammalian subject comprising:
(a) contacting a biological sample of said subject with a reagent that enables measurement of a gene, gene fragment, gene transcript, nucleic acid expression product or protein expression product for human placental lactogen (hPL), a reagent that enables measurement of a gene, gene fragment, gene transcript, nucleic acid expression product or protein expression product for human chorionic gonadotropin (hCG), or a combination of reagents for each of hPL and hCG;
(b) measuring the levels, ratio or pattern of expression of the selected genes, gene fragments, gene transcripts, nucleic acid expression products or protein expression products in the subject's sample and the ratio or pattern of expression levels of the same genes, gene fragments, gene transcripts, nucleic acid expression products or protein expression products in the same biological fluid of a reference or control female mammalian subject having a normal intrauterine pregnancy (IUP),
wherein the presence of, absence of, or changes in expression levels, ratios or patterns of the selected genes, gene fragments, gene transcripts, nucleic acid expression products or protein expression products in relation to the reference or control correlates with a diagnosis of abnormal pregnancy.

2. The method according to claim 1, wherein the abnormal pregnancy is a miscarriage or an ectopic pregnancy.

3. The method according to claim 1, wherein the biological fluid is maternal whole blood, plasma, serum, urine or saliva.

4. The method according to claim 1, wherein the measuring step comprises:
(a) measuring the absolute level of the hPL or hCG messenger ribonucleic acid (mRNA) in the sample and determining its relation to levels of the same selected gene's mRNA of the reference or control;
(b) measuring the absolute levels of hPL mRNA and hCG mRNA in the sample and determining its relation to the same selected gene's mRNA levels of the reference or control;
(c) measuring the ratio of the hPL mRNA to hPL protein in the sample and determining its relation to the same ratio of the reference or control;
(d) measuring the ratio of the hCG mRNA to hCG protein in the sample and determining its relation to the same ratio of the reference or control;
(e) measuring the ratio of the hPL mRNA to hPL protein and the ratio of the hCG mRNA to hCG protein in the sample in the sample and determining the relation to the same ratios of the reference or control;
(f) measuring the absolute level of the hPL or hCG protein expression product in the sample and determining its relation to the same selected gene's protein expression product level of the reference or control;
(g) measuring the absolute levels of hPL protein expression product and hCG protein expression product in the sample and determining the relation to the same selected gene's protein expression product levels of the reference or control;
(h) measuring the ratio of the hPL mRNA to hPL protein in the sample and determining its relation to the same ratio of the reference or control; or
(i) performing any combination of (a) through (h).

5. The method according to claim 1, which further comprises
contacting the biological sample of said subject with a reagent that enables measurement of a gene, gene fragment, gene transcript or expression product of at least one additional biomarker; and
measuring the levels, ratio or pattern of expression of the selected genes, gene fragments, gene transcripts or expression products and the additional biomarker in the subject's sample and determining its relation to the ratio or pattern of expression levels of the same genes, gene fragments, gene transcripts or expression products in the same biological fluid of a reference or control female mammalian subject having a normal intrauterine pregnancy (IUP).

6. The method according to claim 5, further comprising measuring the absolute level of the additional biomarker's mRNA in the sample and determining its relation to the same biomarker's mRNA of the reference or control.

7. The method according to claim 6, further comprising:
(a) measuring the ratio of the additional biomarker's mRNA to the additional biomarker protein expression product in the sample and determining its relation to the same ratio of the reference or control; or
(b) measuring a pattern or signature formed by the levels or ratios of the selected and additional biomarker genes, gene fragments, gene transcripts or expression products of the sample and determining its relation to the pattern formed by the same measurements of the same selected and additional biomarker genes, gene fragments, gene transcripts or expression products of the reference or control.

8. The method according to claim 6, wherein the additional biomarker gene, fragment, transcript or expression product is a biomarker of trophoblast function, a biomarker of endometrial function, a biomarker of angiogenesis, or a biomarkers of corpus luteum function

9. The method according to claim 8, wherein the biomarkers of trophoblast function is activin A, pregnancy-specific beta 1-glocoprotein (SP1), pregnancy-associated plasma protein A (PAPP-A), or inhibin A.

10. The method according to claim 8, wherein the biomarker of endometrial function is glycodelin.

11. The method according to claim 8, wherein the biomarker of angiogenesis is VEGF.

12. The method according to claim 8, wherein the biomarker of corpus luteum function is progesterone or inhibin A.

13. The method according claim 1, wherein the additional biomarker further comprises at least one of the pro-domain or extracellular (EC) domain of ADAM12, Isthmin2 (ISM2), pregnancy specific beta-1 glycoprotein isoform 1 (PSG1), pregnancy specific beta-1 glycoprotein isoform 7 (PSG7), pregnancy specific beta-1 glycoprotein isoform 11 (PSG11), pregnancy specific beta-1 glycoprotein isoform 9 (PSG9), pregnancy specific beta-1 glycoprotein isoform 2 (PSG2); glycoprotein hormones alpha chain precursor (CGA), pappalysin-1 precursor (PAPPA); progestagen-associated endometrial protein (PAEP) or a combination thereof.

14. The method according to claim 1, wherein said change in expression level of each said selected or additional biomarker gene, gene fragment, gene transcript or expression product comprises individually an upregulation in relation to said reference or control or a down regulation in relation to said reference or control.

15. The method according to claim 14, wherein the reduced or absent expression level of hPL and hCG in the subject's sample is indicative of ectopic pregnancy.

16. A diagnostic reagent, panel, or kit for use in diagnosing an abnormal pregnancy in a mammalian subject comprising:
(a) a reagent that enables measurement in a biological sample of an absolute level or ratio of a gene, gene fragment, gene transcript or nucleic acid expression product for human placental lactogen (hPL);
(b) a reagent that enables measurement in a biological sample of an absolute level or ratio of a protein expression product for hPL;
(c) a reagent that enables measurement in a biological sample of an absolute level or ratio of a gene, gene fragment, gene transcript or nucleic acid expression product for human chorionic gonadotropin (hCG);
(d) a reagent that enables measurement in a biological sample of an absolute level or ratio of a protein expression product for hCG;
(e) a reagent that enables measurement in a biological sample of an absolute level or ratio of a gene, gene fragment, gene transcript or nucleic acid expression product of one or more additional biomarker;
(f) a reagent that enables measurement in a biological sample of an absolute level or ratio of a protein expression product for the one or more additional biomarker of (e);
(g) a combination of reagents (a) and (b);
(h) a combination of reagents (c) and (d);
(i) a combination of at least two of reagents (a), (c) and (e);
(j) a combination of at least two of reagents (b), (d) and (f);
(k) a combination of reagents (a), (b), (c) and (d);
(l) a combination of reagents (a), (b), (e) and (f);
(m) a combination of reagents (c), (d), (e) and (f);
(n) a combination of reagents (a) through (f),
wherein at least one said reagent (a) through (n) is optionally associated with a detectable label or a substrate.

17. The reagent, panel or kit of claim 16, wherein the additional biomarker is one or more of activin A, pregnancy-specific beta 1-glocoprotein (SP1), pregnancy-associated plasma protein A (PAPP-A), glycodelin, VEGF, progesterone, inhibin A, glycoprotein hormone alpha chain precursor (CGA), or progestagen-associated endometrial protein (PAEP).

18. The reagent, panel or kit of claim 16, wherein the additional biomarker is the pro-domain or extracellular (EC) domain of ADAM12, Isthmin2 (ISM2), pregnancy specific beta-1 glycoprotein isoform 1 (PSG1), pregnancy specific beta-1 glycoprotein isoform 7 (PSG7), pregnancy specific beta-1 glycoprotein isoform 11 (PSG11), pregnancy specific beta-1 glycoprotein isoform 9 (PSG9), or pregnancy specific beta-1 glycoprotein isoform 2 (PSG2).

19. The reagent, panel or kit according to claim 16, comprising a microarray, a microfluidics card, a chip or a chamber.

20. The reagent, panel or kit according claim 16, which comprises a nucleic acid primer or probe that hybridizes to the gene, gene fragment, gene transcript or nucleic acid expression product of hPL, hCG or an additional biomarker or a ligand that binds to the protein hPL, hCG or the additional biomarker.


SEQUENCE LISTING (9 sequences)

SEQ ID NO: 1 - DNA, 21 bp, Artificial Sequence
  Forward Primer
catgactcccagacctccttc

SEQ ID NO: 2 - DNA, 20 bp, Artificial Sequence
  Reverse Primer
tgcggagcagctctagattg

SEQ ID NO: 3 - DNA, 25 bp, Artificial Sequence
  Probe
ttctgttgcgtttcctccatgttgg

SEQ ID NO: 4 - DNA, 20 bp, Artificial Sequence
  Forward Primer
ctactgccccaccatgaccc

SEQ ID NO: 5 - DNA, 19 bp, Artificial Sequence
  Reverse Primer
tggactcgaagcgcacatc

SEQ ID NO: 6 - DNA, 25 bp, Artificial Sequence
  Probe
cctgcctcaggtggtgtgcaactac

SEQ ID NO: 7 - DNA, 19 bp, Artificial Sequence
  Forward Primer
ccactcctccacccttgac

SEQ ID NO: 8 - DNA, 18 bp, Artificial Sequence
  Reverse Primer
accctgttgctgtagcca

SEQ ID NO: 9 - DNA, 23 bp, Artificial Sequence
  Probe
ttgccctcaacgaccactttgtc